(19) United States
(12) Patent Application Publication
(10) Pub. No.: US 2011/0195950 A1
(43) Pub. Date: Aug. 11, 2011

(54) Organic Compounds

(75) Inventors: Samuel Hintermann, Basel (CH); Konstanze Hurth, St. Genis Pouilly (FR); Marina Tintelnot-Blomley, Maulburg (DE)

(73) Assignee: NOVARTIS AG

(21) Appl. No.: 12/673,365

(22) PCT Filed: Aug. 12, 2008
(86) PCT No.: PCT/EP08/60598

§ 371 Date: Feb. 12, 2010

(30) Foreign Application Priority Data
Aug. 14, 2007 (EP) 07114339.0

(51) Int. Cl. A61K 31/553; C07D 471/04; A61K 31/437; C07D 498/14; A61K 31/5383; C07D 513/14; A61K 31/542; C07D 487/14; A61K 31/551; A61K 31/55; A61P 25/00

(52) U.S. Cl. 514/211.11; 546/82; 514/293; 544/101; 514/229.8; 544/34; 514/224.5; 540/557; 514/220; 540/548; 540/578; 514/215

(57) ABSTRACT

The invention relates to compound of the formula I

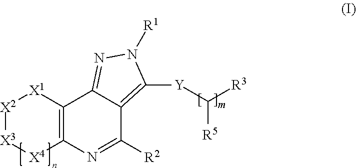

in which R¹ represents an optionally substituted aryl group or an optionally substituted heteroaryl group; R² represents hydrogen or a substituent different from hydrogen; R³ represents an optionally substituted aryl group, cycloalkyl group, heteroaryl group, heterocyclyl group; X¹ represents O, S, NR⁴, CR⁴₂; X² represents O, S, NR⁴, CR⁴₂; X³ represents O, S, NR⁴, CR⁴₂; X⁴ represents O, S, NR⁴, CR⁴₂; R⁴ represents hydrogen or a substituent different from hydrogen; R⁵ represents hydrogen or alkyl; Y represents O or S; m represents 0, 1, 2 or 3; n represents 0, 1, 2 or 3 in free base form or in acid addition salt form; to its preparation, to its use as medicament and to medicaments comprising it.

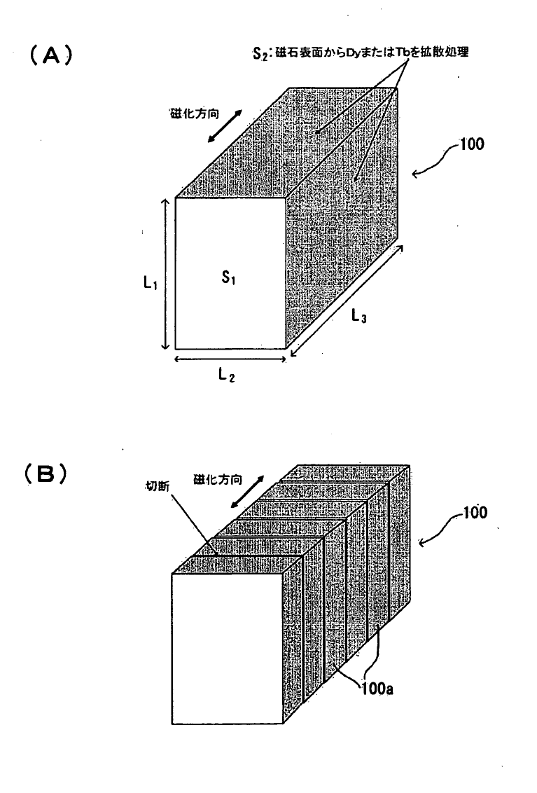

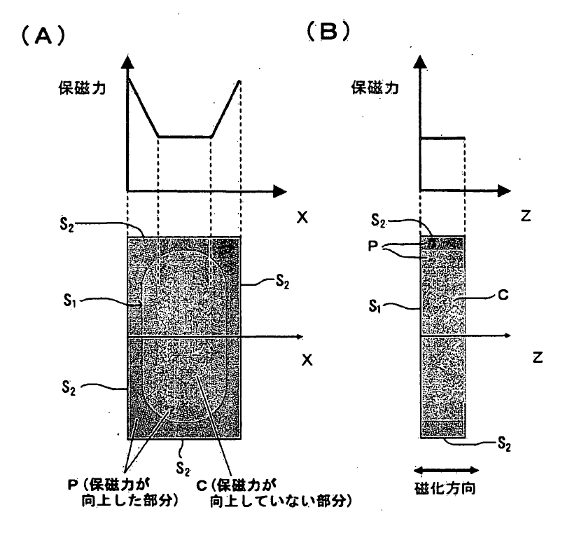

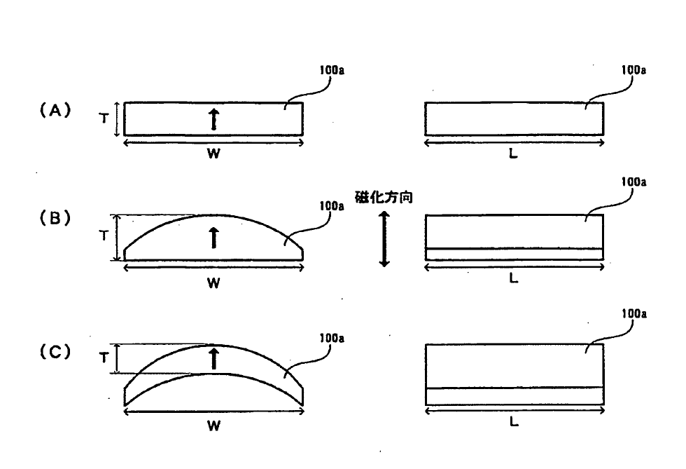

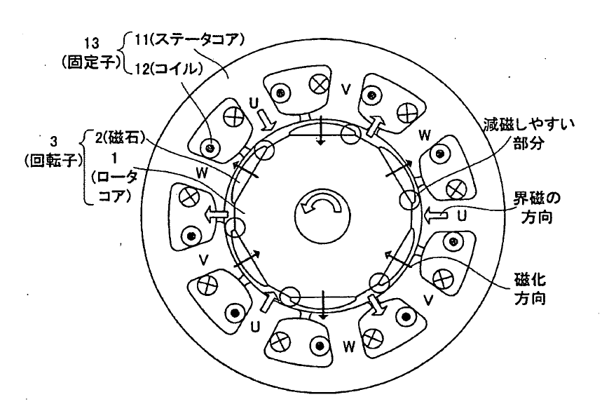

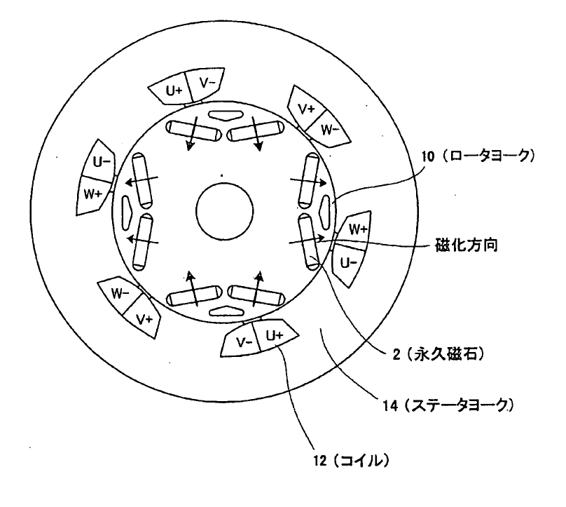

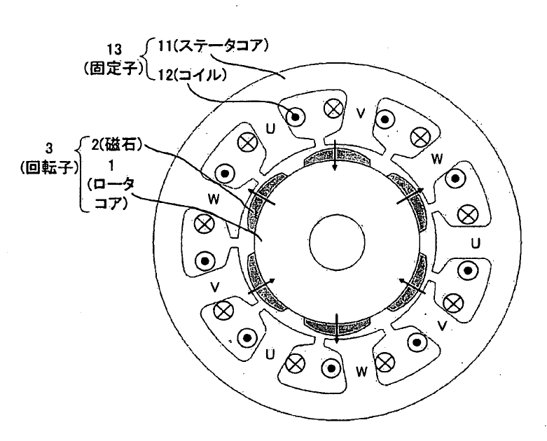

The present invention relates to heterocyclic compounds, to their preparation, to their use as medicaments and to medicaments comprising them.

In a first aspect, the invention relates to a compound of the formula I

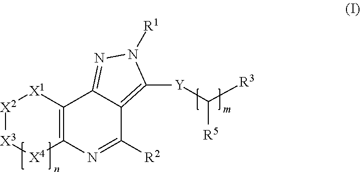

in which
R¹ represents an optionally substituted aryl group or an optionally substituted heteroaryl group;
R² represents hydrogen or a substituent different from hydrogen;
R³ represents an optionally substituted aryl group, cycloalkyl group, heteroaryl group, heterocyclyl group;
X¹ represents O, S, NR⁴, CR⁴₂;
X² represents O, S, NR⁴, CR⁴₂;
X³ represents O, S, NR⁴, CR⁴₂;
X⁴ represents O, S, NR⁴, CR⁴₂;
R⁴ represents hydrogen or a substituent different from hydrogen;
R⁵ represents hydrogen or alkyl;
Y represents O or S;
m represents 0, 1, 2 or 3;
n represents 0, 1, 2 or 3
   in free base form or in acid addition salt form.

If at least one asymmetrical carbon atom is present in a compound of the formula I, such a compound may exist in optically active form or in the form of a mixture of optical isomers, e.g. in the form of a racemic mixture. All optical isomers and their mixtures, including the racemic mixtures, are part of the present invention.

The acid addition salt of compounds of formula I are preferably pharmaceutically acceptable salts. Such salts are known in the field.

It is further understood that, if more than one substituent R⁴ and/or R⁵ are present, each substituent may be independently selected from the list of possible substituents, i.e. one R⁴ may be hydrogen, other substituents R⁴ may be hydrogen or different from hydrogen.

The following general definitions shall apply in this specification, unless otherwise specified:

Halogen (or halo) denotes fluorine, bromine, chlorine or iodine.

Aryl is preferably naphthyl or phenyl, in particular phenyl.

Heterocyclyl represents a saturated or partly saturated ring system containing at least one hetero atom. Preferably, heterocyclyl groups consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), halogen, nitro, cyano, alkyl, alkanediyl, alkenediyl, alkoxy, alkoxyalkyl, alkoxycarbonyl, alkoxycarbonylalkyl, halogenalkyl, aryl, aryloxy, arylalkyl. Heteroaryl represents an aromatic ring system containing at least one hetero atom. Preferably, heteroaryl groups consist of 3 to 11 ring atoms of which 1-3 ring atoms are hetero atoms. Heteroaryl groups may be present as a single ring system or as bicyclic or tricyclic ring systems; preferably as single ring system or as benz-annelated ring system. Bicyclic or tricyclic ring systems may be formed by annelation of two or more rings. A Heterocycle may be substituted by one or more substituents selected from the group consisting of Oxo (═O), halogen, nitro, cyano, alkyl, alkanediyl, alkenediyl, alkoxy, alkoxyalkyl, alkoxycarbonyl, alkoxycarbonylalkyl, halogenalkyl, aryl, aryloxy, arylalkyl. Examples of heterocyclyl and heteroaryl groups include: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline.

Arylalkyl represents an aryl group bound to the molecule via an alkyl group, such as a methyl or ethyl group, preferably phenethyl or benzyl, in particular benzyl. Similarly, cycloalkylalkyl and heterocyclyl represents a cycloalkyl group bound to the molecule via an alkyl group or a heterocyclyl group bound to the molecule via an alkyl group.

Carbon containing groups, moieties or molecules contain 1 to 8, preferably 1 to 6, more preferably 1 to 4, most preferably 1 or 2, carbon atoms. Any non-cyclic carbon containing group or moiety with more than 1 carbon atom is straight-chain or branched.

Halogen-substituted groups and moieties, such as alkyl substituted by halogen, can be mono-, poly- or per-halogenated.

In preferred embodiments, which are preferred independently, collectively or in any combination or sub-combination, the invention relates to a compound of the formula I, in free base form or in acid addition salt form, wherein the substituents are as defined below.
R¹ preferably represents an aryl group or heteroaryl group, said group being unsubstituted or mono-, di-, tri- or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₄₃)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy and —CH═CHCH═CH—, the last-mentioned optional substituent being attached to two adjacent ring carbon atoms of the said aryl group.
R¹ particular preferably represents an aryl group or an heteroaryl group, said group being unsubstituted or mono-, di-, tri- or tetra-substituted, the optional substituent(s) being independently selected from the group consisting of halogen, (C₁₋₈)alkyl, hydroxy, (C₁₋₈)alkoxy, (C₁₋₈)alkoxy substituted by halogen, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, (C₁₋₈)alkoxycarbonyl(C₁₋₈)alkoxy and —CH═CHCH═CH—, the last-mentioned optional substituent being attached to two adjacent ring carbon atoms of the said aryl group.
R¹ very particular preferably represents a phenyl substituted by one or two substituents selected from the group consisting of halo, cyano, C₁-C₄ alkyl, C₁-C₄ alkoxy, such as fluoro, chloro, cyano, methyl, methoxy.
R¹ further very particular preferably represents a heteroaryl group selected from the group consisting of pyridine, 1,2-pyrimidine (pyridazine), 1,3-pyrimidine, 1,4-pyrimidine (pyrazine), said heteroaryl group being optionally substituted by one or two substituents selected from the group consisting of halo, cyano, C₁-C₄ alkyl, C₁-C₄ alkoxy, such as fluoro, chloro, cyano, methyl, methoxy.
R² is preferably selected from the group consisting of hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₆)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋ₛ)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, (C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy.
R² particular preferably represents hydrogen or (C₁₋₄)alkyl.
R² very particular preferably represents hydrogen.
R³ preferably represents an aryl group or a (C₃-C₈)cycloalkyl group, a heteroaryl group with 3 to 8 ring atoms or a heterocyclyl group with 3 to 8 ring atoms;
   wherein said aryl group, (C₃-C₈)cycloalkyl group, heteroaryl group, heterocyclyl group is unsubstituted, mono-substituted, di-substituted or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, dl(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂OC(═O)— and —CH═CHCH═CH—, the four last-mentioned optional substituents in each case being attached to two adjacent ring carbon atoms of the said moiety.
R³ particular preferably represents an aryl group or a (C₃-C₈)cycloalkyl group or a heteraryl group with 5 or 6 ring atoms or a heterocyclyl group with 5 or 6 ring atoms,
   said aryl group being unsubstituted or mono-, di-, tri- or tetra-substituted, the optional substituent(s) being independently selected from the group consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;
   said (C₃-C₈)cycloalkyl being unsubstituted or mono-, di-, tri- or tetra-substituted, the optional substituent(s) being independently selected from the group consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;
   said heteroaryl group being unsubstituted or mono-, di-, tri- or tetra-substituted, the optional substituent(s) being independently selected from the group consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heteroaryl group contains 1-3 nitrogen atoms;
   said heterocyclyl group being unsubstituted or mono-, di-, tri- or tetra-substituted, the optional substituent(s) being independently selected from the group consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclyl group contains 1-3 nitrogen atoms.
Each R⁴ is independently and preferably selected from the group consisting of hydrogen, halogen, (C₁₋₈)alkyl, (C₁₋₈)alkyl substituted by halogen, (C₃₋₈)cycloalkyl, (C₃₋₈)cyclo-(C₁₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, (C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino (C₁₋₈)alkoxy, di(C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy or heteroaryl.
Each R⁴ is independently and particular preferably selected from the group consisting of hydrogen, halogen, (C₁₋₈)alkyl, (C₁₋₈)alkyl substituted by halogen, cyano, (C₁₋₈)alkoxy, amino, (C₁₋₈)alkylamino and di(C₁₋₈)alkylamino with two identical or different (C₁₋₈)alkyl moieties;
Each R⁴ is independently and very particular preferably selected from the group consisting of hydrogen, (C₁₋₄)alkyl or a heteroaryl group selected from the group consisting of pyridyl, pyrimidyl, pyrazinyl, pyrrolyl, imidazolyl, pyrazolyl, triazolyl wherein said heteroaryl group is optionally substituted by one or more (C₁₋₄)alkyl.
R⁵ preferably represents hydrogen or (C₁₋₄)alkyl.
R⁵ particular preferably represents hydrogen or methyl.
Y preferably represents O.
m preferably represents 0, 1 or 2.
m particular preferably represents 1.
n preferably represents 1 or 2.
n particular preferably represents 1.
Each of X¹ to X⁴ preferably and independently represent O or CR⁴₂.
Each of X¹ to X⁴ particular preferably represent CR⁴₂ or each of X¹, X³ and X⁴ particular preferably represent CR⁴₂ and X² particular preferably represents O or each of X¹, X³ and X⁴ particular preferably represent CR⁴₂ and X² particular preferably represents S.

In an advantageous embodiment, the invention relates to a compound of formula IA

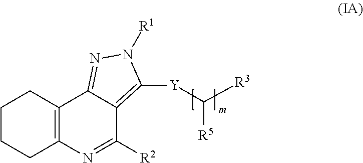

wherein the substituents are as defined for a compound of formula I.

In a further advantageous embodiment, the invention relates to a compound of formula IB

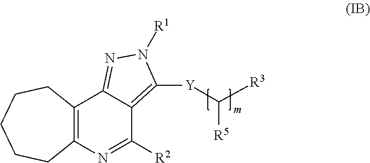

wherein the substituents are as defined for a compound of formula I.

In a further advantageous embodiment, the invention relates to a compound of formula IC

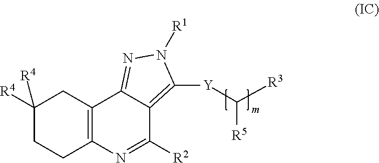

wherein the substituents are as defined for a compound of formula I.

In a further advantageous embodiment, the invention relates to a compound of formula ID

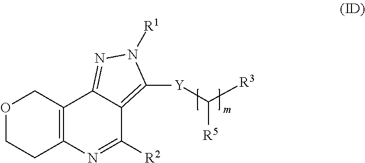

wherein the substituents are as defined for a compound of formula I.

In a further advantageous embodiment, one or two substituents R⁴ are different from hydrogen, while the remaining substituents R⁴ represent hydrogen.

In a further advantageous embodiment, R¹ represents a phenyl substituted in the ortho and/or para-position or in the para position.

In especially preferred embodiments, the invention relates to one or more than one of the compounds of the formula I mentioned in the Examples hereinafter, in free base form or in acid addition salt form.

In a further aspect, the invention relates to a process for the preparation of the compounds of the formula I and their salts, comprising the steps of

A) reacting of a compound of the formula II

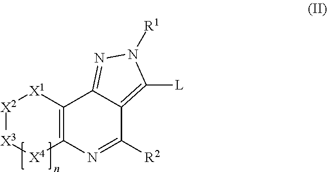

wherein the substituents are as defined for the formula I and L represents a leaving group, such as a halogen, mesylate, tosylate, with a compound of the formula III

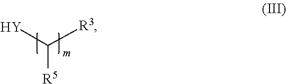

wherein R³, R⁵, m and Y are as defined for R³, R⁵, m and Y in formula I, optionally in the presence of a base, such as a hydride; optionally in the presence of one or more diluents; or
B) reacting of a compound of the formula IV

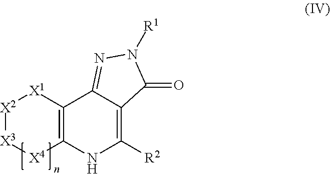

wherein the substituents are as defined for the formula I, with POCl3 followed by a reaction with a compound of the formula III

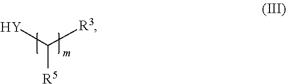

wherein R³, R⁵, m and Y are as defined for R³, R⁵, m and Y in formula I, optionally in the presence of a base, such as a hydride; optionally in the presence of one or more diluents; and
optionally followed by reduction, oxidation or functionalization reaction of the resulting compound of formula I and/or by cleavage of protecting groups optionally present, and
optionally followed by recovering the so obtainable compound of the formula I in free base form or in acid addition salt form.

The reactions can be effected according to conventional methods, for example as described in the Examples. The working-up of the reaction mixtures and the purification of the compounds thus obtainable may be carried out in accordance with known procedures. Acid addition salts may be produced from the free bases in known manner, and vice-versa.

Compounds of the formula I can also be prepared by further conventional processes, e.g. as described in the Examples, which processes are further aspects of the invention.

The starting materials of the formulae II, III and IV are known or may be prepared according to conventional procedures starting from known compounds, for example as described in the Examples.

Compounds of the formula I and their pharmaceutically acceptable acid addition salts, hereinafter sometimes referred to as “agents of the invention”, exhibit valuable pharmacological properties, when tested in vitro and in animals, and are, therefore, useful as active ingredients in medicaments. Agents of the invention have good efficacy as selective ligands for GABA-A receptors, showing desirable GABA-A receptor modulating activities at various receptor subtypes, and, moreover, may possess interesting pharmacokinetic properties, e.g. improved oral bioavailability or enhanced metabolic stability.

Receptors for the major inhibitory neurotransmitter, gamma aminobutyric acid (GABA), are divided into two main classes: GABA-A receptors, which are members of the ligand-gated ion channel superfamily; and GABA-B receptors, which are members of the G-protein coupled receptors superfamily. Since the first cloning of cDNAs encoding individual GABA-A receptor subunits, the number of known mammalian subunits has grown to include at least six alpha subunits, three beta subunits, three gamma subunits, three rho subunits, one delta, one epsilon, one pi, and one phi subunits. With the exception of the rho subunits which form homomultimeric receptor channels, formerly known as GABA-C receptors, it has been indicated, that a pentameric assembly of either alpha and beta subunits or alpha, beta and gamma subunits constitute the minimum requirement for forming a fully functional GABA-A receptor, when expressed by transiently transfecting cDNAs into cells. Functional receptor subtype assemblies, which do exist, include alpha1beta2gamma2, alpha2beta2gamma2 or alpha2beta3gamma2 (alpha2beta2/3gamma2), alpha3beta2/3gamma2 and alpha5beta2gamma2. Delta, epsilon, pi and phi subunits are present only to a minor extent in GABA-A receptor populations. Subtype assemblies containing an alpha1 subunit are present in most areas of the brain and are thought to account for over 40% of GABA-A receptors in the rat. Subtype assemblies containing alpha2 or alpha3 subunits, respectively, are thought to account for about 25% or 17%, respectively, of GABA-A receptors in the rat. Subtype assemblies containing alpha5 subunits are expressed predominantly in the hippocampus and the cortex. A characteristic property of all known GABA-A receptors is the presence of a number of modulatory sites. The benzodiazepine (BZD) binding site is the most explored of these, and it is the site, through which anxiolytic drugs, such as diazepam and midazolam, and hypnotic drugs, such as zolpidem and alpidem, exert their effects. It is believed, that agents acting as BZD agonists at alpha2beta2/3gamma2 and alpha3beta2/3gamma2 subtypes will possess desirable anxiolytic properties. The alpha1-selective GABA-A receptor modulators zolpidem and alpidem are clinically prescribed as hypnotic agents, suggesting that the sedation associated with known anxiolytic drugs, which act at the BZD binding site, is mediated through GABA-A receptors containing the alpha1 subunit. Compounds with inhibitory activity at the BZD site of alpha5beta2gamma2 receptor subtypes are believed to have memory improving effects.

GABA-A receptor modulators show in functional assays a positive modulation of GABA-induced signals. This modulation can be determined in vitro, e.g., at recombinant GABA-A receptors expressed in a mammalian cell line, e.g. by measurement of GABA-A receptor induced changes of the trans-membrane voltage, when using a voltage-sensitive dye and a fluorescence detection system (Adkins, C. E., Pillai, G. V., Kerby, J., Bonnert, T. P., Haldon, C., Mckernan, R. M., Gonzalez, J. E., Oades, K., Whiting, P. J. & Simpson, P. B. [2001]. alpha4beta3delta GABA-A receptors characterized by fluorescence resonance energy transfer-derived measurements of membrane potential. J. Biol. Chem., 276, 38934-38939). In this assay, a modulator compound is pre-applied at different concentrations ranging from 0.1 nM to 10 μM to cells expressing GABA-A receptors and loaded with the voltage-sensitive dye, before, or at the same time as, a sub-maximal concentration of GABA (in the range of from 0.1 to 10 μM) is applied to the cells. The fluorescent signal is correlated with the degree of GABA-A receptor channel opening. This allows the quantification of effects induced by the modulator in a functional manner. By expression of different GABA-A receptor subunit combinations, the differential efficacy of a modulator at different GABA-A receptor variants can be tested. Other functional assays include the electrophysiological recording of Xenopus oocytes or mammalian cells expressing respective receptor variants. In addition, ion flux detectors can be used to functionally study GABA-A receptors in heterologous expression systems. The affinity of a compound to the GABA-A receptor can be measured in radioligand binding experiments using reference ligands containing a radioactive element, e.g., tritiated flumazenil, and intact cells or membrane preparations of cells expressing GABA-A receptors, Activity and selectivity of a GABA-A receptor modulator according to the invention can, e.g., be determined in vitro as follows: A transfected eukaryotic cell line expressing the alpha1, alpha2 or alpha3 subunit of the GABA-A receptor together with a beta and a gamma subunit of the GABA-A receptor is incubated with a voltage-sensitive dye, and the effects of an agonist (typically GABA) or modulator addition are recorded in a fluorimetric plate reader. The opening of the GABA-A receptor channel and the subsequent flux of anions through it changes the trans-membrane voltage of the transfected cells, leading to a change in the fluorescent signal of the voltage-sensitive dye. In the presence of the agent of the invention, a sub-maximal concentration of GABA (e.g. an EC₂₀ or an EC₅₀) added to transfected cells expressing the alpha1, alpha2 or alpha3 subunit of the GABA-A receptor will elicit an at least 50%, preferably an at least 80%, ideally an at least 100%, increase, of the fluorescent signal, compared to the fluorescent signal obtained without the agent of the invention.

In this assay, agents of the invention modulate the GABA-induced response at concentration from about 0.1 to about 10'000 nM. Specifically, the agent of the invention described in Example 1 shows for the alpha1 subunit 37% modulation and for the alpha2 subunit 140% modulation.

In vivo, a GABA-A receptor modulator can be tested in a variety of behavioral or biochemical assays, including, e.g., tests, that assess the anxiolytic-like properties, like the stress-induced hyperthermia test, the light-dark-box assay, the punished drinking (or Vogel-conflict) test, the elevated maze tests or the fear-potentiated startle response test, or tests, that assess the sedative or motor-impairing properties, like the rotarod assays, the test de traction, the primary observation test or the horizontal and vertical locomotion tests.

Due to their GABA-A receptor modulating activities, agents of the invention are useful in the treatment or prevention of a variety of disabilitating psychiatric, psychotic or neurological states, e.g., of conditions, disorders or diseases of the nervous system, that can be modulated or are mediated, fully or in part, by GABA-A receptors. Such conditions, disorders or diseases include anxiety disorders, such as panic disorder with or without agoraphobia, agoraphobia without history of panic disorder, animal or other specific phobias, including social phobias, social anxiety disorder, anxiety, obsessive-compulsive disorder, stress disorders, including post-traumatic or acute stress disorder, or generalized or substance-induced anxiety disorders; neuroses; seizures; epilepsy, especially partial seizures, simple, complex or partial seizures evolving to secondarily generalized seizures or generalized seizures [absence (typical or atypical), myoclonic, clonic, tonic, tonic-clonic or atonic seizures]; convulsions; migraine; affective disorders, including depressive or bipolar disorders, e.g. single-episode or recurrent major depressive disorder, major depression, dysthymic disorder, dysthymia, depressive disorder NOS, bipolar I or bipolar II manic disorder or cyclothymic disorder; psychotic disorders, including schizophrenia; neurodegeneration arising from cerebral ischemia; acute, traumatic or chronic degenerative processes of the nervous system, such as Parkinson's disease, Down's syndrome, senile dementia, cognitive disorders, Alzheimer's disease, Huntington's chorea, amyotrophic lateral sclerosis, multiple sclerosis or fragile X syndrome; attention disorders, e.g. attention deficit hyperactivity disorder; Tourette's syndrome; speech disorders, including stuttering; disorders of the circadian rhythm, e.g. in subjects suffering from the effects of jet lag or shift work; pain or nociception; itch; emesis, including acute, delayed or anticipatory emesis, such as emesis induced by chemotherapy or radiation, motion sickness, or post-operative nausea or vomiting; eating disorders, including anorexia nervosa or bulimia nervosa; premenstrual syndrome; muscle spasm or spasticity, e.g. in paraplegic patients; hearing disorders, e.g. tinnitus or age-related hearing impairment; urinary incontinence; or substance-related disorders, including substance abuse or dependency, including substance, such as alcohol, withdrawal disorders. Agents of the invention may also be useful in enhancing cognition, e.g. in subjects suffering from dementing conditions, such as Alzheimer's disease; as pre-medication prior to anesthesia or minor procedures, such as endoscopy, including gastric endoscopy; or as radioligands or positron emission tomography (PET) ligands in assays for detecting compounds capable of binding to the GABA-A receptor in situ.

For the above-mentioned indications, the appropriate dosage will vary depending on, e.g., the compound employed, the host, the mode of administration and the nature and severity of the condition, disorder or disease. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 10 to about 2000, preferably from about 10 to about 200, mg of an agent of the invention conveniently administered, for example, in divided doses up to four times a day or in sustained release form.

An agent of the invention may be administered by any conventional route, in particular enterally, preferably orally, for example in the form of tablets or capsules, or parenterally, for example in the form of injectable solutions or suspensions.

In accordance with the foregoing, in a further aspect, the invention relates to an agent of the invention, for use as a medicament, e.g. for the treatment or prevention of conditions, disorders or diseases, that can be modulated or are mediated by GABA-A receptors.

In a further aspect, the invention relates to the use of an agent of the invention as active ingredient in a medicament, e.g. for the treatment or prevention of conditions, disorders or diseases, that can be modulated or are mediated by GABA-A receptors.

In a further aspect, the invention relates to a pharmaceutical composition comprising an agent of the invention as active ingredient in association with at least one pharmaceutical carrier or diluent. Such compositions may be manufactured in conventional manner. Unit dosage forms contain, for example, from about 1 to about 1000, preferably from about 1 to about 500, mg of an agent of the invention.

The agents of the invention can be administered alone or as combination with other pharmaceutical agents effective, e.g., in the treatment or prevention of conditions, disorders or diseases mentioned above. Such pharmaceutical combinations may be in the form of a unit dosage form, whereby each unit dosage will comprise a predetermined amount of the two components in admixture with at least one pharmaceutical carrier or diluent. Alternatively, the combination may be in the form of a package containing the two components separately, e.g. a pack or dispenser-device adapted for the concomitant or separate administration of the two active agents, wherein these agents are separately arranged. In a further aspect, the invention relates to such pharmaceutical combinations.

In a further aspect, the invention relates to the use of an agent of the invention for the manufacture of a medicament for the treatment or prevention of conditions, disorders or diseases, that can be modulated or are mediated by GABA-A receptors.

In a further aspect, the invention relates to a method for the treatment or prevention of conditions, disorders or diseases, that can be modulated or are mediated by GABA-A receptors, in a subject in need of such treatment, which comprises administering to such subject a therapeutically effective amount of an agent of the invention.

The following Examples illustrate the invention, but do not limit it.

### Abbreviations

AcOH acetic acid
eq. aqueous
DCM dichloromethane
ESIMS electrospray ionization mass spectrometry
EtOAc ethyl acetate
EtOH ethanol
h hour(s)
HV high vacuum
min minute(s)
MPLC medium pressure liquid chromatography
rt room temperature
soln. solution
HPLC conditions (%=percent by volume)

### Method A (Rt_{A}=Retention Time A)

Agilent 1100 series LC pump; Agilent 1100 series DAD; Agilent 1100 series Col Oven; CTC PAL autosampler; Waters ZQ2000 MS; column Waters XTerra C18 2.5 μm; 3×30 mm; 50° C.; mobile phase: A water 95%+acetonitrile 5%+formic acid 0.2%/B acetonitrile 100%+formic acid 0.2%; injection volume 5 μl; flow 600 μl/min; gradient 5-95% B in 3.5 min; MS parameter 100-900 Da; ESI+cone 17V.

### Method B (Rt_{B}=Retention Time B)

UPLC Waters Acquity; column Acquity HPLC BEH C18 1.7 μm; 2.1×50 mm; gradient: 5 to 100% acetonitrile (0.1% TFA)/water (0.1% TEA), 2 min/100% acetonitrile (0.1% TFA), 0.5 min; flow 0.6 mL/min; 35° C.

### EXAMPLE 1

### 3-(2-chloro-benzyloxy)-2-(4-chloro-phenyl)-6,7,8,9-tetrahydro-2H-pyrazolo[4,3-c]-quinoline

2-(4-Chloro-phenyl)-2,5,6,7,8,9-hexahydro-pyrazolo[4,3-c]quinolin-3-one (1.88 g, 6.27 mmol) is stirred with POCl₃ (10 ml) in a microwave reactor at 110° C. for 1 h. The reaction mixture is poured onto ice, diluted with EtOAc, brought to neutral pH using solid Na₂CO₃ and extracted with EtOAc. The aq. layer is reextracted with EtOAc, the combined organic layers are washed with brine, dried over Na₂SO₄, filtered off and evaporated to give 3-chloro-2-(4-chloro-phenyl)-6,7,8,9-tetrahydro-2H-pyrazolo[4,3-c]quinoline.

To a precooled (0° C.) solution of NaH (206 mg of a 55% dispersion in mineral oil, 4.70 mmol) in THF (4 ml) 2-chloro-benzyl-alcohol (679 mg, 4.71 mmol) is added and the mixture is allowed to warm to rt during a period of 30 min. A solution of 3-chloro-2-(4-chloro-phenyl)-6,7,8,9-tetrahydro-2H-pyrazolo[4,3-c]quinoline (600 mg, 1.89 mmol) in THF (6 ml) is added and the reaction mixture is heated at 60° C. for 3 h, then quenched with water, diluted with EtOAc, dried over Na₂SO₄ and filtered. The filtrate is evaporated off and the remaining solid is purified by flash chromatography (50 g silica gel, gradient 0-5 min DCM:EtOAc 95:5, 5-55 min DCM:EtOAc 95:5 to 65:35). The combined fractions are concentrated in vacuo to give 3-(2-chloro-benzyloxy)-2-(4-chloro-phenyl)-6,7,8,9-tetrahydro-2H-pyrazolo[4,3-c]quinoline which is recrystallized from EtOH. [ESIMS [M+H]⁺=424; HPLC Rt_{A}=2.38 min].

The starting material can be prepared as follows:

### 2-Aminomethylene-malonic acid diethyl ester

2-Ethoxymethylene-malonic acid diethyl ester (20 ml, 100 mmol) and a 10% soln. of ammonia in EtOH (37 ml, 220 mmol) are stirred at rt for 1 h. The mixture is evaporated and dried in HV to give 2-aminomethylene-malonic acid diethyl ester that is used without further purification.

### 2-(Cyclohex-1-enylaminomethylene)-malonic acid diethyl ester

To a solution of cyclohexanone (4.6 ml, 44.3 mmol) in toluene (170 ml) is added 2-aminomethylene-malonic acid diethyl ester (8.3 g, 44.3 mmol) and p-toluenesulfonic acid (305 mg, 1.77 mmol). The reaction mixture is heated to 127° C. for 48 h in a Dean-Stark trap to remove water. The crude mixture is concentrated in vacuo and purified by MPLC (500 g silica gel, eluent cyclohexane:EtOAc 80:20 to 70:30) to give 2-(cyclohex-1-enylaminomethylene)-malonic acid diethyl ester.

### 4-Hydroxy-5,6,7,8-tetrahydro-quinoline-3-carboxylic acid ethyl ester

A soln. of 2-(cyclohex-1-enylaminomethylene)-malonic acid diethyl ester (1 g, 3.74 mmol) in Dowtherm A (10 ml) is stirred in a microwave reactor at 250° C. for 1 h, cooled to rt, diluted with Et₂O and petroleum ether and cooled to 0° C. The precipitating 4-hydroxy-5,6,7,8-tetrahydro-quinoline-3-carboxylic acid ethyl ester is filtered off and dried in HV.

### 4-Chloro-5,6,7,8-tetrahydro-quinoline-3-carboxylic acid ethyl ester

A solution of 4-hydroxy-5,6,7,8-tetrahydro-quinoline-3-carboxylic acid ethyl ester (3.85 g, 17.0 mmol) in POCl₃ (17 ml) is stirred in a microwave reactor at 120° C. for 1 h. The reaction mixture is poured onto ice, diluted with EtOAc, brought to neutral pH using solid Na₂CO₃ and 4M NaOH soln, and extracted twice with EtOAc. The combined organic layers are washed with brine, dried over Na₂SO₄, treated with charcoal and filtered over hyflo. The filtrate is evaporated off to yield 4-chloro-5,6,7,8-tetrahydro-quinoline-3-carboxylic acid ethyl ester.

### 2-(4-Chloro-phenyl)-2,5,6,7,8,9-hexahydro-pyrazolo[4,3-c]quinolin-3-one

To a solution of 4-chloro-5,6,7,8-tetrahydro-quinoline-3-carboxylic acid ethyl ester (1.66 g, 6.93 mmol) and (4-chloro-phenyl)-hydrazine hydrochloride (2.53 g, 13.8 mmol) in n-butanol (20 ml) is added NEt₃ (3.39 ml, 24.2 mmol) and the reaction mixture is heated to reflux for 1 h at 125° C. After cooling to rt, the mixture is concentrated in vacuo, diluted with Et₂O, the precipitate is filtered off, washed with Et₂O and H₂O and dried in HV to yield 2-(4-chlorophenyl)-2,5,6,7,8,9-hexahydro-pyrazolo[4,3-c]quinolin-3-one.

### EXAMPLES 2 TO 80

The compounds of Table 1 are obtainable in a manner analogous to that described in ex. 1.

TABLE 1

| | | ESIMS | |
|---|---|---|---|
| Ex. | Compound | [M + H]⁺ | HPLC |
| 1 | 3-(2-Chloro-benzyloxy)-2-(4-chloro-phenyl)-6,7,8,9- | 424 | Rt_{A} = |
| | tetrahydro-2H-pyrazolo[4,3-c]quinoline | | 2.38 |
| 2 | 3-(2-Chloro-benzyloxy)-2-(4-chloro-phenyl)-2,6,7,8,9,10- | 438 | Rt_{A} = |
| | hexahydro-1,2,5-triaza-cyclohepta[e]indene | | 2.40 |
| 3 | 3-(2-Chloro-benzyloxy)-2-(4-fluoro-phenyl)-2,6,7,8,9,10- | 422 | Rt_{A} = |
| | hexahydro-1,2,5-triaza-cyclohepta[e]indene | | 2.28 |
| 4 | 3-(2-Chloro-benzyloxy)-phenyl-2,6,7,8,9,10- | 404 | Rt_{A} = |
| | hexahydro-1,2,5-triaza-cyclohepta[e]indene | | 2.26 |
| 5 | 3-(4-Chloro-benzyloxy)-2-(4-chloro-phenyl)-6,7,8,9- | 424 | Rt_{A} = |
| | tetrahydro-2H-pyrazolo[4,3-c]quinoline | | 2.47 |
| 6 | 3-(2-Chloro-benzyloxy)-2-(4-chloro-phenyl)-2,6,7,9- | 426 | Rt_{A} = |
| | tetrahydro-8-oxa-1,2,5-triaza-cyclopenta[a]naphthalene | | 2.24 |
| 7 | 3-(2-Chloro-benzyloxy)-2-(4-chloro-phenyl)-8,8-dimethyl- | 452 | Rt_{A} = |
| | 6,7,8,9-tetrahydro-2H-pyrazolo[4,3-c]quinoline | | 2.59 |
| 8 | 3-(4-Chloro-benzyloxy)-2-(4-chloro-phenyl)-8,8-dimethyl- | 452 | Rt_{A} = |
| | 6,7,8,9-tetrahydro-2H-pyrazolo[4,3-c]quinoline | | 2.59 |
| 9 | 3-(2-Chloro-benzyloxy)-2-(4-methoxy-phenyl)- | 434 | Rt_{A} = |
| | 2,6,7,8,9,10-hexahydro-1,2,5-triaza-cyclohepta[e]indene | | 2.32 |
| 10 | 3-(4-Chloro-benzyloxy)-2-(4-methoxy-phenyl)- | 434 | Rt_{A} = |
| | 2,6,7,8,9,10-hexahydro-1,2,5-triaza-cyclohepta[e]indene | | 2.35 |
| 11 | 2-(4-Chloro-phenyl)-3-(2-fluoro-benzyloxy)-6,7,8,9- | 408 | Rt_{A} = |
| | tetrahydro-2H-pyrazolo[4,3-c]quinoline | | 2.27 |
| 12 | 2-(4-Chloro-phenyl)-3-(4-fluoro-benzyloxy)-6,7,8,9- | 408 | Rt_{A} = |
| | tetrahydro-2H-pyrazolo[4,3-c]quinoline | | 2.29 |
| 13 | 2-(4-Chloro-phenyl)-3-cyclopropylmethoxy-6,7,8,9- | 354 | Rt_{A} = |
| | tetrahydro-2H-pyrazolo[4,3-c]quinoline | | 2.17 |
| 14 | 2-(4-Chloro-phenyl)-3-cyclobutylmethoxy-6,7,8,9- | 368 | Rt_{A} = |
| | tetrahydro-2H-pyrazolo[4,3-c]quinoline | | 2.29 |
| 15 | 2-(4-Chloro-phenyl)-3-cyclopentylmethoxy-6,7,8,9- | 382 | Rt_{A} = |
| | tetrahydro-2H-pyrazolo[4,3-c]quinoline | | 2.37 |
| 16 | 2-(4-Chloro-phenyl)-3-cyclohexylmethoxy-6,7,8,9- | 396 | Rt_{A} = |
| | tetrahydro-2H-pyrazolo[4,3-c]quinoline | | 2.47 |
| 17 | 2-(4-Chloro-phenyl)-3-(3-methyl-butoxy)-6,7,8,9- | 370 | Rt_{A} = |
| | tetrahydro-2H-pyrazolo[4,3-c]quinoline | | 2.36 |
| 18 | 2-(4-Chloro-phenyl)-3-(6-chloro-pyridin-3-ylmethoxy)- | 425 | Rt_{A} = |
| | 6,7,8,9-tetrahydro-2H-pyrazolo[4,3-c]quinoline | | 2.09 |
| 19 | 3-[2-(4-Chloro-phenyl)-6,7,8,9-tetrahydro-2H- | 415 | Rt_{A} = |
| | pyrazolo[4,3-c]quinolin-3-yloxymethyl]-benzonitrile | | 2.20 |
| 20 | 4-[2-(4-Chloro-phenyl)-6,7,8,9-tetrahydro-2H- | 415 | Rt_{A} = |
| | pyrazolo[4,3-c]quinolin-3-yloxymethyl]-benzonitrile | | 2.23 |
| 21 | 2-(4-Chloro-phenyl)-3-(pyridin-4-ylmethoxy)-6,7,8,9- | 391 | Rt_{A} = |
| | tetrahydro-2H-pyrazolo[4,3-c]quinoline | | 1.60 |
| 22 | 2-(4-Chloro-phenyl)-3-(2-trifluoromethyl-benzyloxy)- | 458 | Rt_{A} = |
| | 6,7,8,9-tetrahydro-2H-pyrazolo[4,3-c]quinoline | | 2.29 |
| 23 | 2-(4-Chloro-phenyl)-3-(3-trifluoromethyl-benzyloxy)- | 458 | Rt_{A} = |
| | 6,7,8,9-tetrahydro-2H-pyrazolo[4,3-c]quinoline | | 2.32 |
| 24 | 2-(4-Chloro-phenyl)-3-(4-trifluoromethyl-benzyloxy)- | 458 | Rt_{A} = |
| | 6,7,8,9-tetrahydro-2H-pyrazolo[4,3-c]quinoline | | 2.37 |
| 25 | 3-(3-Chloro-benzyloxy)-2-(4-chloro-phenyl)-6,7,8,9- | 425 | Rt_{A} = |
| | tetrahydro-2H-pyrazolo[4,3-c]quinoline | | 2.31 |
| 26 | 3-(2-Chloro-benzyloxy)-2-(2-fluoro-phenyl)-6,7,8,9- | 408 | Rt_{A} = |
| | tetrahydro-2H-pyrazolo[4,3-c]quinoline | | 2.04 |
| 27 | 3-(2-Chloro-benzyloxy)-2-(4-methoxy-phenyl)-6,7,8,9- | 420 | Rt_{A} = |
| | tetrahydro-2H-pyrazolo[4,3-c]quinoline | | 2.11 |
| 28 | 3-(2-Chloro-benzyloxy)-2-(4-ethoxy-phenyl)-2,6,7,8,9,10- | 448 | Rt_{A} = |
| | hexahydro-1,2,5-triaza-cyclohepta[e]indene | | 2.34 |
| 29 | 4-[3-(2-Chloro-benzyloxy)-6,7,8,9-tetrahydro- | 415 | Rt_{A} = |
| | pyrazolo[4,3-c]quinolin-2-yl]-benzonitrile | | 2.11 |
| 30 | 4-(3-Cyclopropylmethoxy-6,7,8,9-tetrahydro- | 345 | Rt_{A} = |
| | pyrazolo[4,3-c]quinolin-2-yl)-benzonitrile | | 1.86 |
| 31 | 3-(2-Fluoro-benzyloxy)-2-(4-fluoro-phenyl)-2,6,7,9- | 394 | Rt_{A} = |
| | tetrahydro-8-oxa-1,2,5-triaza-cyclopenta[a]naphthalene | | 1.95 |
| 32 | 3-(2-Fluoro-benzyloxy)-2-pyridin-4-yl-2,6,7,8,9,10- | 389 | Rt_{A} = |
| | hexahydro-1,2,5-triaza-cyclohepta[e]indene | | 1.80 |
| 33 | 3-(2-Fluoro-benzyloxy)-2-pyridin-2-yl-2,6,7,8,9,10- | 389 | Rt_{A} = |
| | hexahydro-1,2,5-triaza-cyclohepta[e]indene | | 1.92 |
| 34 | 2-(4-Chloro-phenyl)-3-(2-fluoro-benzyloxy)-2,6,7,9- | 426 | Rt_{A} = |
| | tetrahydro-8-thia-1,2,5-triaza-cyclopenta[a]naphthalene | | 2.25 |
| 35 | 2-(4-Chloro-phenyl)-3-cyclopentylmethoxy-2,6,7,9- | 384 | Rt_{A} = |
| | tetrahydro-8-oxa-1,2,5-triaza-cyclopenta[a]naphthalene | | 2.21 |
| 36 | 4-(3-Cyclopropylmethoxy-6,7,8,9-tetrahydro- | 345 | Rt_{A} = |
| | pyrazolo[4,3-c]quinolin-2-yl)-benzonitrile | | 1.86 |
| 37 | 3-(2-Fluoro-benzyloxy)-2-phenyl-2,6,7,9-tetrahydro-8- | 376 | Rt_{A} = |
| | oxa-1,2,5-triaza-cyclopenta[a]naphthalene | | 1.91 |
| 38 | 3-(2-Fluoro-benzyloxy)-2-pyridin-3-yl-2,6,7,8,9,10- | 389 | Rt_{A} = |
| | hexahydro-1,2,5-triaza-cyclohepta[e]indene | | 1.89 |
| 39 | 3-(2-Fluoro-benzyloxy)-2-pyridin-2-yl-6,7,8,9-tetrahydro- | 375 | Rt_{A} = |
| | 2H-pyrazolo[4,3-c]quinoline | | 1.81 |
| 40 | 2-(4-Chloro-phenyl)-3-cyclopropylmethoxy-2,6,7,9- | 356 | Rt_{A} = |
| | tetrahydro-8-oxa-1,2,5-triaza-cyclopenta[a]naphthalene | | 1.97 |
| 41 | 3-(2-Chloro-phenoxy)-2-(4-chloro-phenyl)-6,7,8,9- | 410 | Rt_{A} = |
| | tetrahydro-2H-pyrazolo[4,3-c]quinoline | | 2.33 |
| 42 | 4-[3-(2-Fluoro-benzyloxy)-6,7,8,9-tetrahydro- | 399 | Rt_{A} = |
| | pyrazolo[4,3-c]quinolin-2-yl]-benzonitrile | | 2.00 |
| 43 | 2-(4-Chloro-phenyl)-3-(2-fluoro-benzyloxy)-2,6,7,9- | 410 | Rt_{A} = |
| | tetrahydro-8-oxa-1,2,5-triaza-cyclopenta[a]naphthalene | | 2.13 |
| 44 | 2-(4-Chloro-phenyl)-3-[1-(2-fluoro-phenyl)-ethoxy]- | 422 | Rt_{A} = |
| | 6,7,8,9-tetrahydro-2H-pyrazolo[4,3-c]quinoline | | 2.35 |
| 45 | 3-(4-Chloro-phenoxy)-2-(4-chloro-phenyl)-6,7,8,9- | 410 | Rt_{A} = |
| | tetrahydro-2H-pyrazolo[4,3-c]quinoline | | 2.30 |
| 46 | 2-(4-Chloro-phenyl)-3-(2-fluoro-phenoxy)-2,6,7,9- | 396 | Rt_{A} = |
| | tetrahydro-8-oxa-1,2,5-triaza-cyclopenta[a]naphthalene | | 2.16 |
| 47 | 2-(4-Chloro-phenyl)-3-[1-(2-chloro-phenyl)-ethoxy]- | 438 | Rt_{A} = |
| | 6,7,8,9-tetrahydro-2H-pyrazolo[4,3-c]quinoline | | 2.39 |
| 48 | 2-(4-Chloro-phenyl)-3-(2-fluoro-phenoxy)-6,7,8,9- | 394 | Rt_{A} = |
| | tetrahydro-2H-pyrazolo[4,3-c]quinoline | | 2.19 |
| 49 | 2-(4-Chloro-phenyl)-3-(4-fluoro-phenoxy)-6,7,8,9- | 394 | Rt_{A} = |
| | tetrahydro-2H-pyrazolo[4,3-c]quinoline | | 2.17 |
| 50 | 2-(4-Chloro-phenyl)-3-[1-(4-fluoro-phenyl)-ethoxy]- | 422 | Rt_{A} = |
| | 6,7,8,9-tetrahydro-2H-pyrazolo[4,3-c]quinoline | | 2.31 |
| 51 | 2-(4-Chloro-phenyl)-3-[1-(4-chloro-phenyl)-ethoxy]- | 438 | Rt_{A} = |
| | 6,7,8,9-tetrahydro-2H-pyrazolo[4,3-c]quinoline | | 2.48 |
| 52 | 2-(4-Chloro-phenyl)-3-cyclopentyloxy-6,7,8,9-tetrahydro- | 368 | Rt_{A} = |
| | 2H-pyrazolo[4,3-c]quinoline | | 2.25 |
| 53 | 3-(2-Chloro-phenoxy)-2-(4-chloro-phenyl)-2,6,7,9- | 412 | Rt_{A} = |
| | tetrahydro-8-oxa-1,2,5-triaza-cyclopenta[a]naphthalene | | 2.24 |
| 54 | 2-(4-Chloro-phenyl)-3-cyclopentyloxy-2,6,7,9-tetrahydro- | 370 | Rt_{A} = |
| | 8-oxa-1,2,5-triaza-cyclopenta[a]naphthalene | | 2.07 |
| 55 | 2-(4-Chloro-phenyl)-3-cycloheptyloxy-6,7,8,9-tetrahydro- | 396 | Rt_{A} = |
| | 2H-pyrazolo[4,3-c]quinoline | | 2.42 |
| 56 | 2-(4-Chloro-phenyl)-3-cyclohexyloxy-6,7,8,9-tetrahydro- | 382 | Rt_{A} = |
| | 2H-pyrazolo[4,3-c]quinoline | | 2.32 |
| 57 | 2-(4-Chloro-phenyl)-3-cyclobutoxy-6,7,8,9-tetrahydro- | 354 | Rt_{A} = |
| | 2H-pyrazolo[4,3-c]quinoline | | 2.16 |
| 58 | 2-(4-Chloro-phenyl)-3-(2-morpholin-4-yl-ethoxy)-6,7,8,9- | 413 | Rt_{A} = |
| | tetrahydro-2H-pyrazolo[4,3-c]quinoline | | 1.43 |
| 59 | 2-(4-Chloro-phenyl)-3-(2,4-difluoro-benzyloxy)-6,7,8,9- | 426 | Rt_{A} = |
| | tetrahydro-2H-pyrazolo[4,3-c]quinoline | | 2.31 |
| 60 | 2-(4-Chloro-phenyl)-3-(tetrahydro-furan-3-ylmethoxy)- | 384 | Rt_{A} = |
| | 6,7,8,9-tetrahydro-2H-pyrazolo[4,3-c]quinoline | | 2.00 |
| 61 | 2-(4-Chloro-phenyl)-3-oxiranylmethoxy-6,7,8,9- | 356 | Rt_{A} = |
| | tetrahydro-2H-pyrazolo[4,3-c]quinoline | | 1.92 |
| 62 | 2-(4-Chloro-phenyl)-3-(2-morpholin-4-yl-ethoxy)-2,6,7,9- | 415 | Rt_{A} = |
| | tetrahydro-8-oxa-1,2,5-triaza-cyclopenta[a]naphthalene | | 1.25 |
| 63 | 2-(4-Chloro-phenyl)-3-(tetrahydro-furan-2-ylmethoxy)- | 384 | Rt_{A} = |
| | 6,7,8,9-tetrahydro-2H-pyrazolo[4,3-c]quinoline | | 2.06 |
| 64 | 2-(4-Chloro-phenyl)-3-(tetrahydro-furan-2-ylmethoxy)- | 386 | Rt_{A} = |
| | 2,6,7,9-tetrahydro-8-oxa-1,2,5-triaza- | | 1.84 |
| | cyclopenta[a]naphthalene | | |
| 65 | 2-(4-Chloro-phenyl)-3-[2-(4-methyl-piperazin-1-yl)- | 428 | Rt_{A} = |
| | ethoxy]-2,6,7,9-tetrahydro-8-oxa-1,2,5-triaza- | | 1.31 |
| | cyclopenta[a]naphthalene | | |
| 66 | 2-(4-Chloro-phenyl)-3-(tetrahydro-furan-3-ylmethoxy)- | 386 | Rt_{A} = |
| | 2,6,7,9-tetrahydro-8-oxa-1,2,5-triaza- | | 1.83 |
| | cyclopenta[a]naphthalene | | |
| 67 | 2-(4-Chloro-phenyl)-3-(tetrahydro-pyran-2-ylmethoxy)- | 398 | Rt_{A} = |
| | 6,7,8,9-tetrahydro-2H-pyrazolo[4,3-c]quinoline | | 2.20 |
| 68 | 2-(4-Chloro-phenyl)-3-(oxetan-2-ylmethoxy)-6,7,8,9- | 370 | Rt_{A} = |
| | tetrahydro-2H-pyrazolo[4,3-c]quinoline | | 1.93 |
| 69 | 2-(4-Chloro-phenyl)-3-(tetrahydro-pyran-4-ylmethoxy)- | 398 | Rt_{A} = |
| | 6,7,8,9-tetrahydro-2H-pyrazolo[4,3-c]quinoline | | 2.09 |
| 70 | 2-(4-Chloro-phenyl)-3-cyclopropylmethoxy-8-pyrimidin-2- | 433 | Rt_{B} = |
| | yl-6,7,8,9-tetrahydro-2H-pyrazolo[4,3- | | 1.34 |
| | c][1,6]naphthyridine | | |
| 71 | 2-(4-Chloro-phenyl)-3-(1-methyl-piperidin-4-yloxy)- | 397 | Rt_{A} = |
| | 6,7,8,9-tetrahydro-2H-pyrazolo[4,3-c]quinoline | | 1.48 |
| 72 | 2-(4-Chloro-phenyl)-3-(tetrahydro-pyran-4-yloxy)-6,7,8,9- | 384 | Rt_{A} = |
| | tetrahydro-2H-pyrazolo[4,3-c]quinoline | | 2.02 |
| 73 | 2-(4-Chloro-phenyl)-3-(1-methyl-piperidin-2-ylmethoxy)- | 411 | Rt_{A} = |
| | 6,7,8,9-tetrahydro-2H-pyrazolo[4,3-c]quinoline | | 1.51 |
| 74 | 2-(4-Chloro-phenyl)-3-(1-methyl-piperidin-3-ylmethoxy)- | 411 | Rt_{A} = |
| | 6,7,8,9-tetrahydro-2H-pyrazolo[4,3-c]quinoline | | 1.50 |
| 75 | 2-(4-Chloro-phenyl)-3-(tetrahydro-pyran-3-yloxy)-6,7,8,9- | 384 | Rt_{A} = |
| | tetrahydro-2H-pyrazolo[4,3-c]quinoline | | 2.11 |
| 76 | 2-(4-Chloro-phenyl)-3-(1-methyl-piperidin-3-yloxy)- | 397 | Rt_{A} = |
| | 6,7,8,9-tetrahydro-2H-pyrazolo[4,3-c]quinoline | | 1.48 |
| 77 | 2-(4-Chloro-phenyl)-3-(1-methyl-piperidin-4-ylmethoxy)- | 407 | Rt_{A} = |
| | 2H-pyrazolo[4,3-c]quinoline | | 1.56 |
| 78 | 3-(2-Chloro-benzyloxy)-2-(4-chloro-phenyl)-8-pyrimidin- | 503 | Rt_{B} = |
| | 2-yl-6,7,8,9-tetrahydro-2H-pyrazolo[4,3- | | 1.46 |
| | c][1,6]naphthyridine | | |
| 79 | 2-(4-Chloro-phenyl)-3-(2-pyrrolidin-1-yl-ethoxy)-2H- | 393 | Rt_{A} = |
| | pyrazolo[4,3-c]quinoline | | 1.50 |
| 80 | 2-(4-Chloro-phenyl)-3-(1-cyclopentyl-ethoxy)-6,7,8,9- | 396 | Rt_{A} = |
| | tetrahydro-2H-pyrazolo[4,3-c]quinoline | | 2.46 |

1. A compound of the formula I 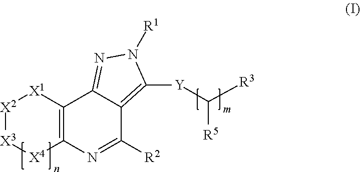
in which
R¹ represents an optionally substituted aryl group or an optionally substituted heteroaryl group;
R² represents hydrogen or a substituent different from hydrogen;
R³ represents an optionally substituted aryl group, cycloalkyl group, heteroaryl group, heterocyclyl group;
X¹ represents O, S, NR⁴, CR⁴₂;
X² represents O, S, NR⁴, CR⁴₂;
X³ represents O, S, NR⁴, CR⁴₂;
X⁴ represents O, S, NR⁴, CR⁴₂;
R⁴ represents hydrogen or a substituent different from hydrogen;
R⁵ represents hydrogen or alkyl;
Y represents O or S;
m represents 0, 1, 2 or 3;
n represents 0, 1, 2 or 3
in free base form or in acid addition salt form.

2. A compound of formula I according to claim 1 wherein
R¹ represents an aryl group or heteroaryl group, said group being unsubstituted or mono-, di-, tri- or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkyl, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, morpholino(C₁₋₈)alkoxy, piperidino(C₁₋₈)alkoxy, pyrrolidino(C₁₋₈)alkoxy, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy and —CH═CHCH═CH—, the last-mentioned optional substituent being attached to two adjacent ring carbon atoms of the said aryl group;
R² represents hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₃)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy;
R³ represents an aryl group or a (C₃-C₈)cycloalkyl group, or a heteroaryl group with 3 to 8 ring atoms or a heterocyclyl group with 3 to 8 ring atoms;
wherein said aryl group, (C₃-C₈)cycloalkyl group, heteroaryl group, heterocyclyl group is unsubstituted, mono-substituted, di-substituted or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈) cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl (C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈) alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkyl amino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkyl carbonyl(C₁₋₈)alkyl, formyl(C₁₋₈)alkoxy, (C₁₋₈)alkylcarbonyl(C₁₋₈)alkoxy, (C₁₋₈)alkoxy carbonyl, (C₁₋₈)alkoxyoarbonylcory, (C₁₋₈)alkoxycarbonyl(C₁₋₄₃)alkyl, (C₁₋₈)alkoxy carbonyl(C₁₋₈)alkoxy, —OCH₂O—, —C(O)OCH₂—, —CH₂OC(O)— and —CH:CHCH:CH—, the four last-mentioned optional substituents in each case being attached to two adjacent ring carbon atoms of the said group.
X¹ represents NR⁴, CR⁴₂;
X² represents NR⁴, CR⁴₂;
X³ represents NR⁴, CR⁴₂;
X⁴ represents NR⁴, CR⁴₂;
R⁴ represents, independently from each other, hydrogen, halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈) cycloalkoxy, (C₃₋₈)cycloalkoxy(C₁₋₈)alkyl, (C₃₋₈)cycloalkyl(C₁₋₈)alkoxy, (C₃₋₈) cycloalkoxy(C₁₋₈)alkoxy, aryl, aryl(C₁₋₈)alkyl, aryloxy, aryloxy(C₁₋₈)alkyl, aryl(C₁₋₈) alkoxy, aryloxy(C₁₋₈)alkoxy, cyano, nitro, carboxy, carbamyl, hydroxy, (C₁₋₈) alkoxy, (C₁₋₈)alkoxy(C₁₋₈)alkoxy, (C₁₋₈)alkoxy substituted by halogen, (C₁₋₈)alkoxy (C₁₋₈)alkyl, (C₁₋₈)alkylthio, (C₁₋₈)alkylthio(C₁₋₈)alkyl, (C₁₋₈)alkylsulfinyl, (C₁₋₈)alkyl sulfinyl(C₁₋₈)alkyl, (C₁₋₈)alkylsulfonyl, (C₁₋₈)alkylsulfonyl(C₁₋₈)alkyl, amino, (C₁₋₈) alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy or heteroaryl;
R⁵ represents hydrogen or (C₁₋₄)alkyl;
Y represents O or S;
m represents 0, 1 or 2;
n represents 1 or 2;
in free base form or in acid addition salt form.

3. A process for the preparation of a compound of the formula I as defined in claim 1, in free base form or in acid addition salt form, comprising the steps of
A) reacting of a compound of the formula II 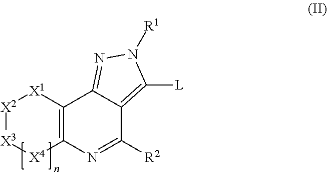
wherein the substituents are as defined for the formula I in claim 1 and L represents a leaving group, such as a halogen, tosylate, mesylate, with a compound of the formula III 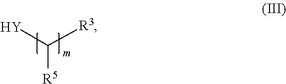
wherein R³, R⁵, m and Y are as defined for R³, R⁵, m and Y in formula I in claim 1, optionally in the presence of a base, such as a hydride; optionally in the presence of one or more diluents; or
B) reacting of a compound of the formula IV 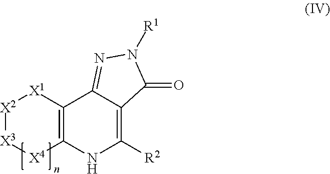
wherein the substituents are as defined for the formula I in claim 1, with POCl3 followed by a reaction with a compound of the formula III 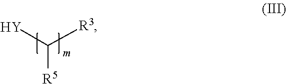
wherein R³, R⁵, m and Y are as defined for R³, R⁵, m and Yin formula I in claim 1, optionally in the presence of a base, such as a hydride; optionally in the presence of one or more diluents; and
optionally followed by reduction, oxidation or functionalisation reaction of the resulting compound of formula I and/or by cleavage of protecting groups optionally present, and
optionally followed by recovering the so obtainable compound of the formula I in free base form or in acid addition salt form.

4.-6. (canceled)

7. A method for the treatment, prevention or delay of progression of a condition, disease or disorder, that can be modulated or is mediated by GABA-A receptors, comprising administering to a subject in need thereof a therapeutically effective amount of a compound of the formula I as defined in claim 1, in free form or in pharmaceutically acceptable salt form.

8. A pharmaceutical composition comprising a compound of the formula I as defined in claim 1, in free form or in pharmaceutically acceptable salt form, as active ingredient, and a pharmaceutical carrier or diluent.

9. A pharmaceutical composition comprising a therapeutically effective amount of a compound of the formula I as defined in claim 1, in free form or in pharmaceutically acceptable salt form, and a second drug substance.
